(19) United States
(12) Patent Application Publication
(10) Pub. No.: US 2007/0265587 A1
(43) Pub. Date: Nov. 15, 2007

(54) Vacuum container and method of using thereof

(76) Inventor: Hsin-Yu Chen, Taichung City (TW)

Correspondence Address: BIRCH STEWART KOLASCH & BIRCH, PO BOX 747, FALLS CHURCH, VA, 22040-0747, US

(21) Appl. No.: 11/431,565

(22) Filed: May 11, 2006

(51) Int. Cl. A61M 1/00

(52) U.S. Cl. 604/319; 604/320

(57) ABSTRACT

A vacuum container has a cap with a recess port on the top thereof, and an aperture on the bottom side of the recess port to receive a strut type button. The button has a retaining flange on a lower side to press the bottom wall of the cap. There are air vents around the aperture. The button further has a membrane extending outwards to cover the air vents. A vacuum cylinder can be coupled with the recess port to withdraw the air in the container to form a vacuum condition. The invention integrates the vacuuming and pressure releasing structures into one body at a smaller size, thus can be used on a small container. A plurality of smaller containers can be housed in a vacuum chest. While the vacuum chest is vacuuming, the smaller containers are also being vacuumed at the same time.

### FIELD OF THE INVENTION

The present invention relates to a vacuum container and particularly to a structure that integrates functions of vacuuming, pressure releasing and vacuum indication in one body to be adaptable to smaller containers.

### BACKGROUND OF THE INVENTION

A conventional vacuum preservation can as shown in FIGS. 1 and 2 mainly includes a can 1 with a upward opening and a cap 2 located on the top of the can 1. The cap 2 has a hollow base 3 and a lid 4 coupling on the top of the base 3. The base 3 contains two suction ducts 5 communicating with the can 1. The two suction ducts 5 further are coupled with a drawing bar 6 which can be moved reciprocally to withdraw the air in the can 1 so that the interior of the can 1 becomes a vacuum condition to prevent goods held in the can 1 from in contact with the air, thereby to extend the preservation time period of the goods. When in use to fetch the goods held in the can 1, depress a pressure release button 7 on the lid 4, then external air can enter the can 1 to enable the cap 2 to be removed for fetching the goods. To enable users to easily recognize whether the interior of the can 1 is in a vacuum condition, an indication button 8 is installed on the base 3. While the drawing bar 6 is moved to withdraw the air from the can 1, the indication button 8 is sunk under pressure into an aperture 9 formed on the lid 4, thereby the vacuum condition of the can 1 can be determined.

The can 1 and cap 2 mentioned above have to allocate a significant space to accommodate the suction ducts 5, drawing bar 6, pressure release button 7 and indication button 8. In practice, many small cans (such as medicine cans) have to be vacuumed to extend the preservation time period of goods contained inside. As the cans are too small, to contain the vacuuming facilities previously discussed in the cap is difficult. As a result, the aforesaid conventional structure cannot be widely used on small cans.

### SUMMARY OF THE INVENTION

The primary object of the present invention is to provide a vacuum container that has a cap with a recess port formed on the top, an aperture on the bottom side of the recess port, and a strut type button held in the aperture. The button has a retaining flange on a lower side to press the bottom wall of the cap. The recess port further has air vents around the aperture. The button has a membrane on the periphery to cover the air vents. A cap type vacuum indication member is located above the membrane. The button has a through hole in the center. During vacuuming the vacuum indication member sinks under pressure to enable users to recognize the vacuum condition. A vacuum cylinder can be coupled with the recess port to withdraw the air in the container to form a vacuum condition. Such a structure integrates the vacuuming, pressure releasing and vacuum indication functions into one body so that it can be adopted to small containers to extend the preservation time period of goods held inside.

Another object of the invention is to provide a vacuum container using method that includes a vacuum chest to house a plurality of small vacuum containers mentioned above. While the vacuum chest is vacuuming, the membrane in the small vacuum containers also is driven by airflow so that the small vacuum containers also are being vacuumed. The vacuum chest may be electrically driven or a larger vacuum container constructed as previously discussed.

The foregoing, as well as additional objects, features and advantages of the invention will be more readily apparent from the following detailed description, which proceeds with reference to the accompanying drawings.

### BRIEF DESCRIPTION OF THE DRAWINGS

FIGS. 1 and 2 are schematic views of a conventional vacuum preservation can.

FIG. 3 is an exploded view of a first embodiment of the invention.

FIG. 4 is a sectional view of the first embodiment of the invention.

FIG. 5A is a schematic view of the first embodiment of the invention in a use condition.

FIG. 5B is a schematic view of the first embodiment of the invention in another use condition.

FIG. 6A is a sectional view of a second embodiment of the invention.

FIG. 6B is a schematic view of the second embodiment of the invention in a vacuum condition.

FIG. 7 is a schematic view of the invention adopted on a larger container to house smaller containers.

FIG. 8 is a schematic view of an electrically driven vacuum chest of the invention in a use condition.

FIG. 9 is a schematic view of a control means of an electrically driven vacuum chest of the invention.

FIG. 10 is a schematic view of an electrically driven vacuum chest of the invention in a use condition for housing containers.

FIG. 11A is a schematic view of an electrically driven vacuum chest of the invention in another use condition for housing containers.

FIG. 11B is a fragmentary enlarged view according to FIG. 11A.

FIG. 12 is a schematic view of an electrically driven vacuum chest of the invention in yet another use condition for housing containers.

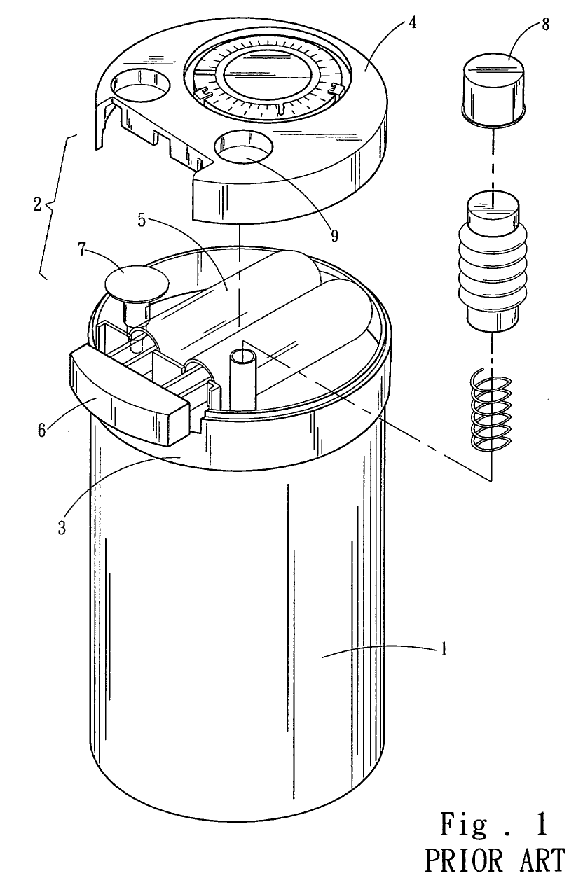

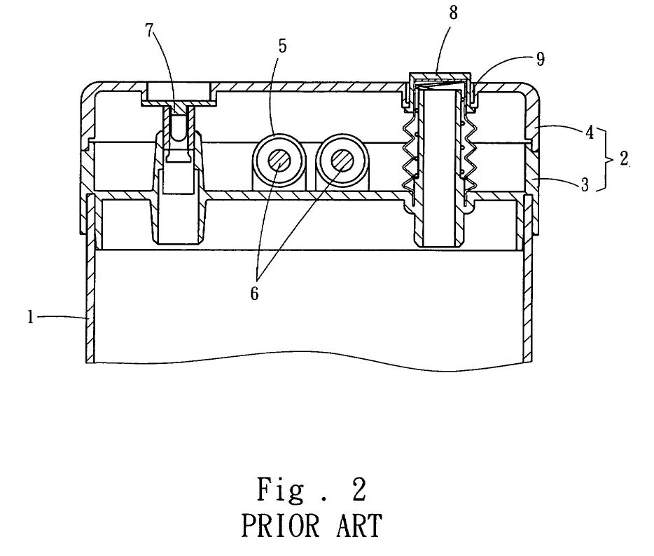

### DETAILED DESCRIPTION OF THE PREFERRED EMBODIMENTS

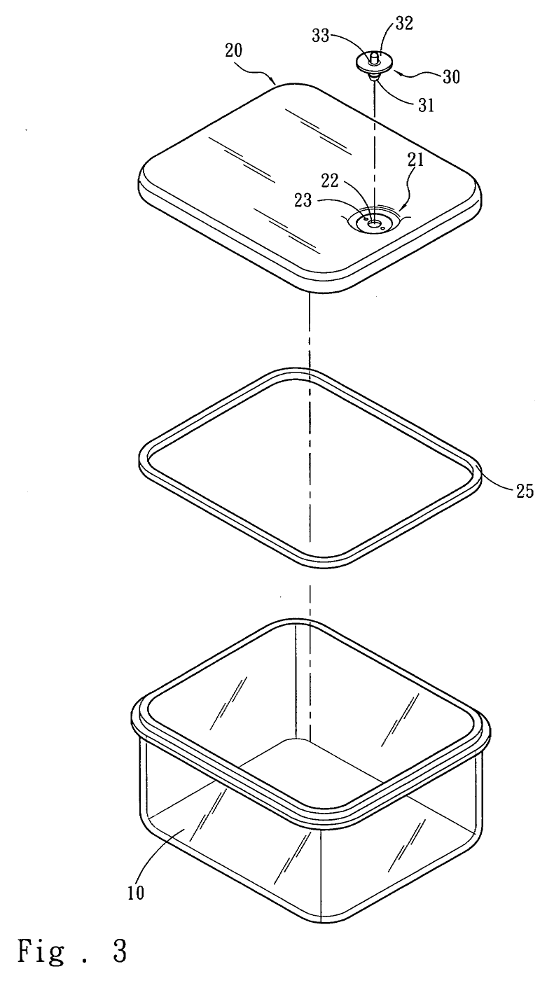

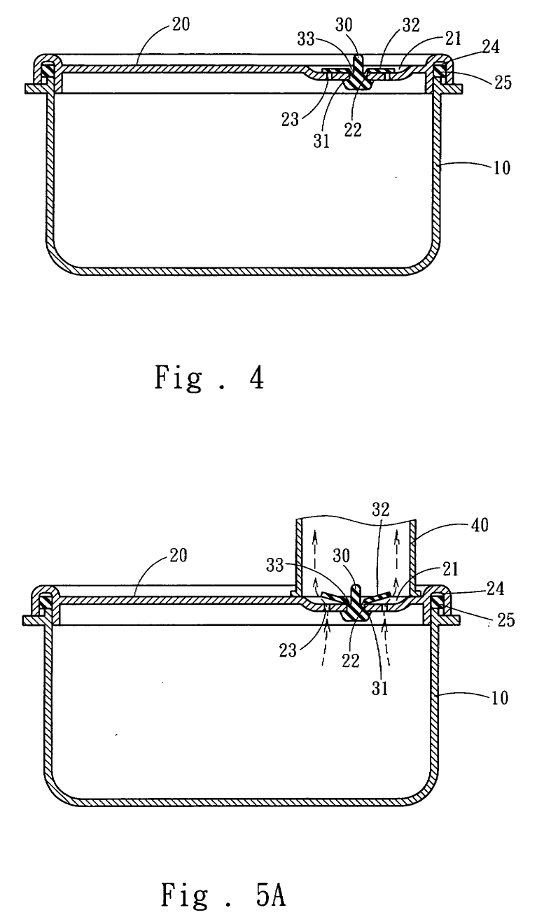

Please referring to FIGS. 3 and 4, the vacuum container according to the invention includes a container 10 and a cap 20 coupling on an opening formed on the top of the container 10. The cap 20 has a recess port 21 on the top that has an aperture 22 in the center to communicate inside and outside. The recess port 21 further has a plurality of air vents 23 around the aperture 22. The cap 20 has an annular groove 24 on the bottom to hold a leakage-preventing ring 25.

A strut type button 30 is provided to be inserted into the aperture 22 in a up and down manner. The button 30 has a retaining flange 31 on the bottom at a greater diameter and a membrane 32 extended outwards from a middle portion thereof. The membrane 32 has a diameter larger enough to cover the air vents 23. The membrane 32 and the button 30 are joined at a juncture which has an indented bending rim 33.

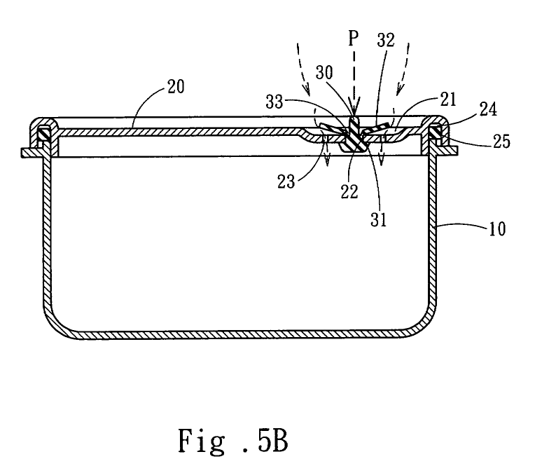

By means of the construction set forth above, the button 30 can be inserted into the cap 20 by force through the aperture 22, with the retaining flange 31 of the bottom 30 pressing the bottom wall of the cap 20 to form an anchoring condition. When in use, the cap 20 covers the opening of the container 10, and a vacuum cylinder 40 is covered the recess port 21 to withdraw air. The membrane 32 will be lifted due to the suction force. Hence the air in the container 10 can escape through the air vents 23 of the recess port 21 as shown in FIG. 5A. When the interior of the container 10 becomes vacuum, the leakage-preventing ring 25 on the bottom of the cap 20 can prevent air leakage. The membrane 32 of the bottom 30 is in contact closely with the upper side of the air vents 23 under pressure to maintain the interior of the container 10 in the vacuum condition. Therefore the preservation time period of goods held in the container 10 can be extended.

To open the cap 20 to fetch the goods in the container 10, depress the button 30 as shown in FIG. 5B. The membrane 32 and the button 30 are joined at a juncture which has an indented bending rim 33. The bending rim 33 allows the membrane 32 to be lifted easily and moved away from the air vents 23 on the surface of the recess port 21, therefore external air can enter the container 10 to allow the cap 20 to be removed easily.

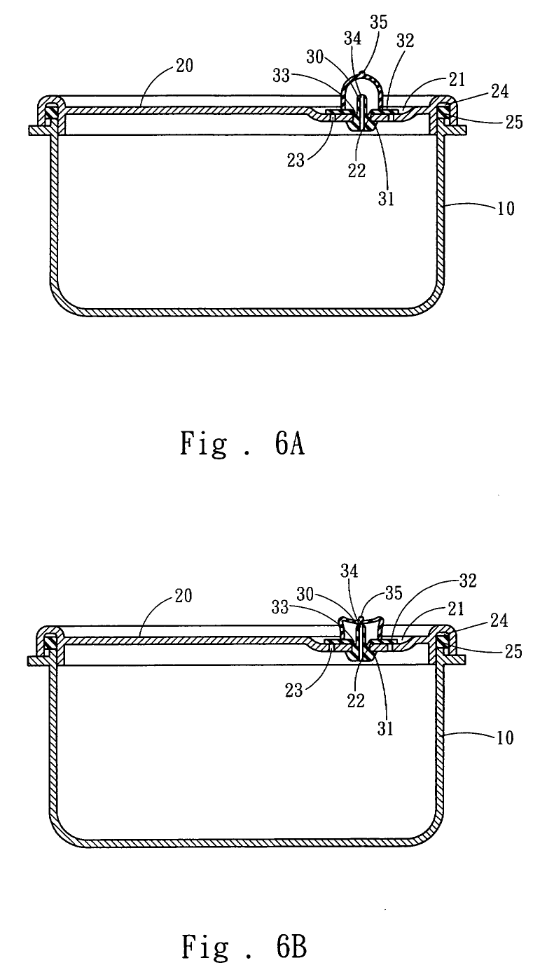

Refer to FIG. 6A for a second embodiment of the invention. It differs from the previous embodiment by adding a pliable vacuum indication member 35 above the membrane 32. The vacuum indication member 35 is a hollow cap with an opening directing downwards to cover the upper side of the membrane 32. The top of the vacuum indication member 35 is spaced from the top of the button 30 so that the top of the vacuum indication member 35 can sink to a lower position. The button 30 further has a through hole 34 running through the center in a up and down manner.

By means of the structure previously discussed, when the container 10 is in the vacuum condition, the pliable vacuum indication member 35 sinks due to the external pressure is greater than the internal pressure of the container 10 as shown in FIG. 6B. Therefore users can see and recognize whether the interior of the container 10 is in the vacuum condition.

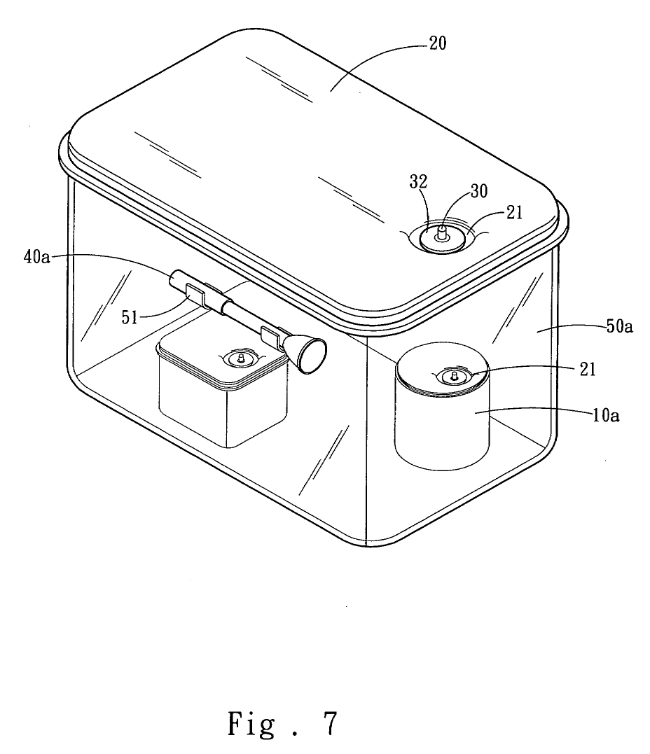

Refer to FIG. 7 for a first embodiment to implement the invention. It has a larger vacuum chest 50a formed by the container 10 to house a plurality of smaller containers 10a (such as medicine cans). When in use, the vacuum cylinder 40 is coupled with the recess port 21 of the vacuum chest 50a to withdraw the air to vacuum the interior of the vacuum chest 50a. As airflow is circulated around the smaller containers 10a housed in the vacuum chest 50a and air pressure decreases, the air inside the smaller containers 10a also drives the membrane 32 and flows outwards at the same time. As a result, the smaller containers 10a also are vacuumed. Thus the vacuuming operation of the larger vacuum chest 50a also can vacuum a plurality of smaller containers 10a. It is a convenient vacuum operation.

In addition, the inner side of the vacuum chest 50a has two holding portions 51 to hold another vacuum cylinder 40a to be used by the smaller containers 10a.

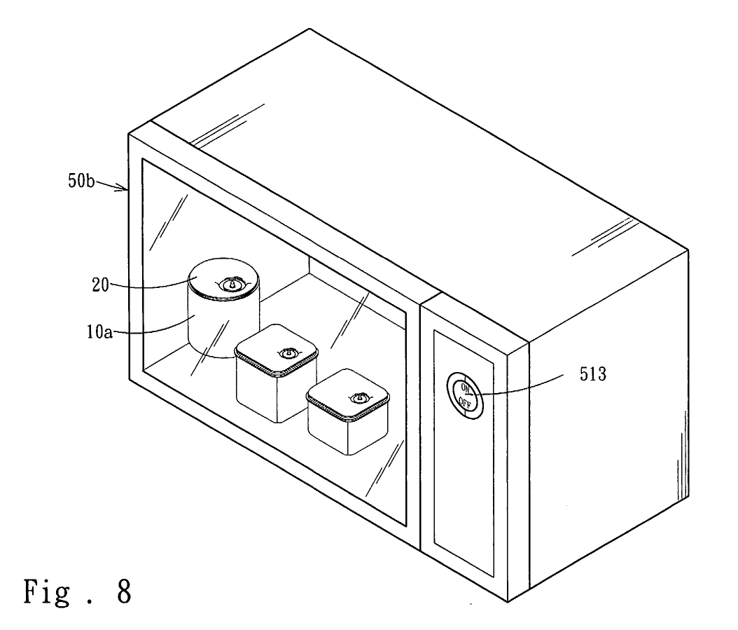

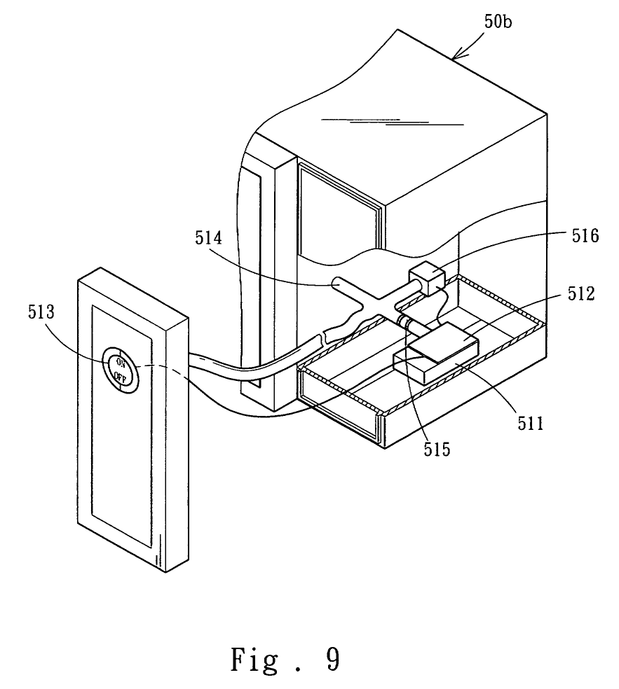

Refer to FIGS. 8 and 9 for a second embodiment to implement the invention. It has an electrically driven vacuum chest 50b formed in a rectangular shape that includes a control means on one side. The control means includes a motor 511 on one side of the interior to be activated by a control panel 512. The vacuum chest 50b has a front face panel which has a button 513 electrically connected to the control panel 512. There is a piping 514 with one end leading to the interior of the vacuum chest 50b and other end connecting to the motor 511. The piping 514 has a check valve 515 to control air intake and discharge. The control panel 512 is connected to a sensor 516. When the button is in an ON condition, the motor 511 is activated to perform vacuuming operation for the vacuum chest 50b. The sensor 516 can also detect the vacuum condition of the vacuum chest 50b to stop operation of the motor 511. On the other hand, when the button 513 is in an OFF condition, the piping 514 forms a free passage to allow external air to flow into the vacuum chest 50b to release the pressure inside. Then the chest lid can be opened to allow the containers 10a to be removed.

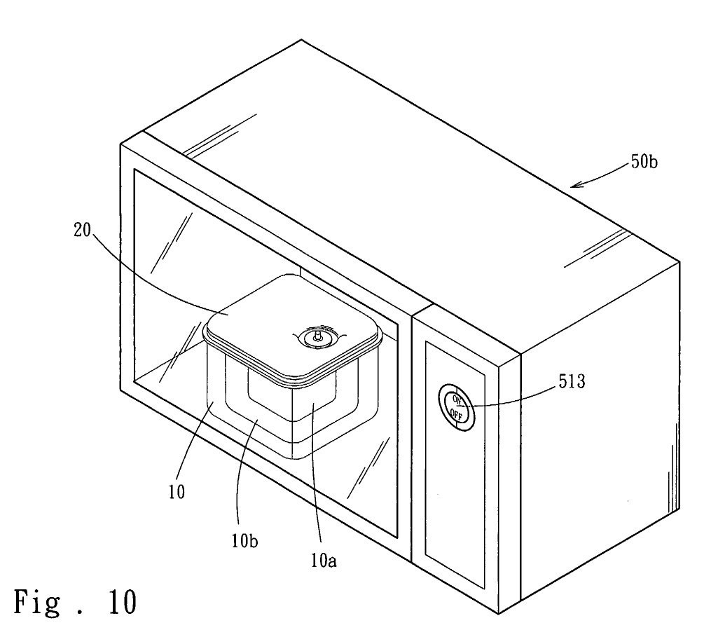

Referring to FIG. 10, the containers 10 and 10a held in the vacuum chest 50b can be in various sizes so that the smaller container 10a can be held in the intermediate container 10b, and the intermediate container 10b can be held in the larger container 10.

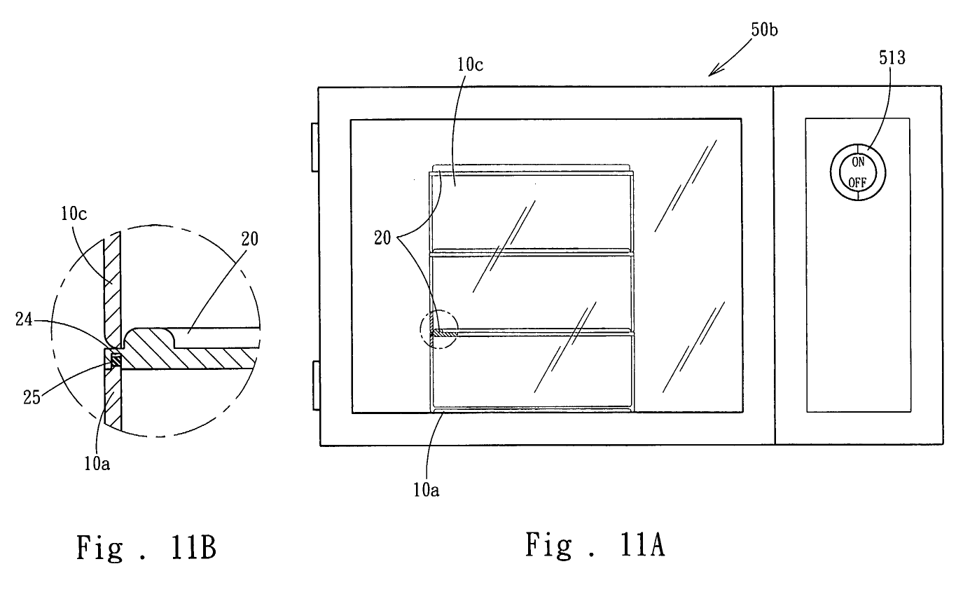

Referring to FIGS. 11A and 11B, a plurality of smaller containers 10a and 10c may also be stacked and held in the vacuum chest 50b. The container 10a at the bottom has a cap 20 on the top thereof to serve as the base of the upper container 10c. The container 10c at the top also has another cap 20. By stacking a plurality of the containers, when the vacuum chest 50b is vacuuming, the interior of the stacked containers 10a and 10c can also be vacuumed.

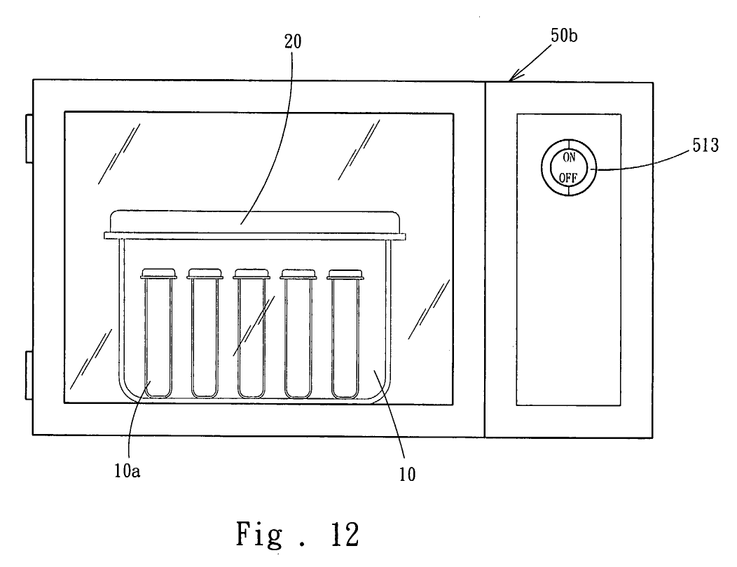

Refer to FIG. 12 for another implementing condition of the invention. A large container 10 houses a plurality of smaller containers 10a (such as medicine cans), then the large container 10 is held in a vacuum chest 50b. When the vacuum chest 50b is vacuuming, the smaller containers 10a (or intermediate containers) and the large container 10 are under pressure and become vacuum.

In short, the invention can provide the following benefits:

1. The cap 20 of the container 10 is structured to contain integrated functions of vacuuming, pressure releasing and vacuum indication at a minimum size to reduce occupying space on the top side of the cap 20. It not only can be adopted to the large container 10, also can be used on the smaller containers 10a (such as medicine cans).

2. A plurality of smaller containers 10a can be housed in the larger vacuum chest 50a or 50b to extend the preservation time period of goods held in the containers 10a. While the vacuum chest 50a or 50b is vacuuming, the containers 10, 10a, 10b and 10c housed inside also are vacuumed. Operation is simpler and can be used to hold goods that require to be stored in a very dry condition.

While the preferred embodiments of the invention have been set forth for the purpose of disclosure, modifications of the disclosed embodiments of the invention as well as other embodiments thereof may occur to those skilled in the art. Accordingly, the appended claims are intended to cover all embodiments which do not depart from the spirit and scope of the invention.

What is claimed is:

1. A vacuum container, comprising:
a container and a cap coupling with the container, the cap having an aperture on the top thereof, at least one air vent around the aperture and an annular groove on the bottom thereof to hold a leakage-preventing ring inside; and
a strut type button which is inserted into the aperture and has a retaining flange on a lower side to press the bottom surface of the cap and a membrane extending outwards from a middle portion thereof to cover the air vent.

2. The vacuum container of claim 1, wherein the cap has a recess port on the top, the aperture being located on the recess port.

3. The vacuum container of claim 1, wherein the button and the membrane are joined at a juncture which has an indented bending rim.

4. The vacuum container of claim 1, wherein the button has a through hole in the center, the membrane being covered by a hollow cap type vacuum indication member from above that has a top spacing from the top of the button so that the top of the vacuum indication member is sinkable.

5. A method of using a vacuum container, comprising:
providing a vacuum chest for vacuuming; and
housing at least one vacuum container in the vacuum chest.

6. The method of claim 5, wherein the container includes a cap coupling on an opening of the container, the cap including:
an aperture, at least one air vent around the aperture and an annular groove on the bottom thereof to hold a leakage-preventing ring inside; and
a strut type button which is inserted into the aperture of the cap and has a retaining flange on a lower side to press the bottom surface of the cap and a membrane extending outwards from a middle portion thereof to cover the air vent.

7. The method of claim 5, wherein the vacuum chest is electrically driven and includes a control means, the control means including:
a motor;
a control panel to control activation of the motor;
a button on a face panel of the vacuum chest to be electrically connected to the control panel by a circuit;
a piping which has one end leading to the interior of the vacuum chest and other end connecting to the motor;
a check valve located on the piping to control air intake and discharge; and
a sensor connecting to the control panel to detect vacuum conditions of the vacuum chest and control stop operation of the motor.

8. The method of claim 5, wherein the vacuum container housed in the vacuum chest includes a smaller container held in an intermediate container which is held in a larger vacuum container.

9. The method of claim 5, wherein the vacuum container housed in the vacuum chest includes a plurality of smaller containers held in the vacuum chest in a stacking manner, the bottom and the top containers respectively having a cap coupling on the top thereof, the cap of the bottom container serving as a base of the container in the middle.

10. The method of claim 5, wherein the vacuum container housed in the vacuum chest includes a larger container housing a plurality of smaller containers.
